SEQUENCE LISTING (30 sequences)

SEQ ID NO: 1 - DNA, 15 bp, synthetic construct
  source 1..15: mol_type = other DNA
ccaaaaaagaaggaa

SEQ ID NO: 2 - DNA, 21 bp, synthetic construct
  source 1..21: mol_type = other DNA
ccaaaaaagaagagaaaggta

SEQ ID NO: 3 - DNA, 6696 bp, synthetic construct
  source 1..6696: mol_type = other DNA
agcgaagaaagcttattcgagtcttctccacagaagatggagtacgaaattacaaactactcagaaagacatacagaacttccaggtcatttcattggcctcaatacagtagataaactagaggagtccccgttaagggactttgttaagagtcacggtggtcacacggtcatatccaagatcctgatagcaaataatggtattgccgccgtgaaagaaattagatccgtcagaaaatgggcatacgagacgttcggcgatgacagaaccgtccaattcgtcgccatggccaccccagaagatctggaggccaacgcagaatatatccgtatggccgatcaatacattgaagtgccaggtggtactaataataacaactacgctaacgtagacttgatcgtagacatcgccgaaagagcagacgtagacgccgtatgggctggctggggtcacgcctccgagaatccactattgcctgaaaaattgtcccagtctaagaggaaagtcatctttattgggcctccaggtaacgccatgaggtctttaggtgataaaatctcctctaccattgtcgctcaaagtgctaaagtcccatgtattccatggtctggtaccggtgttgacaccgttcacgtggacgagaaaaccggtctggtctctgtcgacgatgacatctatcaaaagggttgttgtacctctcctgaagatggtttacaaaaggccaagcgtattggttttcctgtcatgattaaggcatccgaaggtggtggtggtaaaggtatcagacaagttgaacgtgaagaagatttcatcgctttataccaccaggcagccaacgaaattccaggctcccccattttcatcatgaagttggccggtagagcgcgtcacttggaagttcaactgctagcagatcagtacggtacaaatatttccttgttcggtagagactgttccgttcagagacgtcatcaaaaaattatcgaagaagcaccagttacaattgccaaggctgaaacatttcacgagatggaaaaggctgccgtcagactggggaaactagtcggttatgtctctgccggtaccgtggagtatctatattctcatgatgatggaaaattctactttttagaattgaacccaagattacaagtcgagcatccaacaacggaaatggtctccggtgttaacttacctgcagctcaattacaaatcgctatgggtatccctatgcatagaataagtgacattagaactttatatggtatgaatcctcattctgcctcagaaatcgatttcgaattcaaaactcaagatgccaccaagaaacaaagaagacctattccaaagggtcattgtaccgcttgtcgtatcacatcagaagatccaaacgatggattcaagccatcgggtggtactttgcatgaactaaacttccgttcttcctctaatgtttggggttacttctccgtgggtaacaatggtaatattcactccttttcggactctcagttcggccatatttttgcttttggtgaaaatagacaagcttccaggaaacacatggttgttgccctgaaggaattgtccattaggggtgatttcagaactactgtggaatacttgatcaaacttttggaaactgaagatttcgaggataacactattaccaccggttggttggacgatttgattactcataaaatgaccgctgaaaagcctgatccaactcttgccgtcatttgcggtgccgctacaaaggctttcttagcatctgaagaagcccgccacaagtatatcgaatccttacaaaagggacaagttctatctaaagacctactgcaaactatgttccctgtagattttatccatgagggtaaaagatacaagttcaccgtagctaaatccggtaatgaccgttacacattatttatcaatggttctaaatgtgatatcatactgcgtcaactatctgatggtggtcttttgattgccataggcggtaaatcgcataccatctattggaaagaagaagttgctgctacaagattatccgttgactctatgactactttgttggaagttgaaaacgatccaacccagttgcgtactccatcccctggtaaattggttaaattcttggtggaaaatggtgaacacattatcaagggccaaccatatgcagaaattgaagttatgaaaatgcaaatgcctttggtttctcaagaaaatggtatcgtccagttattaaagcaacctggttctaccattgttgcaggtgatatcatggctattatgactcttgacgatccatccaaggtcaagcacgctctaccatttgaaggtatgctgccagattttggttctccagttatcgaaggaaccaaacctgcctataaattcaagtcattagtgtctactttggaaaacattttgaagggttatgacaaccaagttattatgaacgcttccttgcaacaattgatagaggttttgagaaatccaaaactgccttactcagaatggaaactacacatctctgctttacattcaagattgcctgctaagctagatgaacaaatggaagagttagttgcacgttctttgagacgtggtgctgttttcccagctagacaattaagtaaattgattgatatggccgtgaagaatcctgaatacaaccccgacaaattgctgggcgccgtcgtggaaccattggcggatattgctcataagtactctaacgggttagaagcccatgaacattctatatttgtccatttcttggaagaatattacgaagttgaaaagttattcaatggtccaaatgttcgtgaggaaaatatcattctgaaattgcgtgatgaaaaccctaaagatctagataaagttgcgctaactgttttgtctcattcgaaagtttcagcgaagaataacctgatcctagctatcttgaaacattatcaaccattgtgcaagttatcttctaaagtttctgccattttctctactcctctacaacatattgttgaactagaatctaaggctaccgctaaggtcgctctacaagcaagagaaattttgattcaaggcgctttaccttcggtcaaggaaagaactgaacaaattgaacatatcttaaaatcctctgttgtgaaggttgcctatggctcatccaatccaaagcgctctgaaccagatttgaatatcttgaaggacttgatcgattctaattacgttgtgttcgatgttttacttcaattcctaacccatcaagacccagttgtgactgctgcagctgctcaagtctatattcgtcgtgcttatcgtgcttacaccataggagatattagagttcacgaaggtgtcacagttccaattgttgaatggaaattccaactaccttcagctgcgttctccacctttccaactgttaaatctaaaatgggtatgaacagggctgtttctgtttcagatttgtcatatgttgcaaacagtcagtcatctccgttaagagaaggtattttgatggctgtggatcatttagatgatgttgatgaaattttgtcacaaagtttggaagttattcctcgtcaccaatcttcttctaacggacctgctcctgatcgttctggtagctccgcatcgttgagtaatgttgctaatgtttgtgttgcttctacagaaggtttcgaatctgaagaggaaattttggtaaggttgagagaaattttggatttgaataagcaggaattaatcaatgcttctatccgtcgtatcacatttatgttcggttttaaagatgggtcttatccaaagtattatacttttaacggtccaaattataacgaaaatgaaacaattcgtcacattgagccggctttggccttccaactggaattaggaagattgtccaacttcaacattaaaccaattttcactgataatagaaacatccatgtctacgaagctgttagtaagacttctccattggataagagattctttacaagaggtattattagaacgggtcatatccgtgatgacatttctattcaagaatatctgacttctgaagctaacagattgatgagtgatatattggataatttagaagtcaccgacacttcaaattctgatttgaatcatatcttcatcaacttcattgcggtgtttgatatctctccagaagatgtcgaagccgccttcggtggtttcttagaaagatttggtaagagattgttgagattgcgtgtttcttctgccgaaattagaatcatcatcaaagatcctcaaacaggtgccccagtaccattgcgtgccttgatcaataacgtttctggttatgttatcaaaacagaaatgtacaccgaagtcaagaacgcaaaaggtgaatgggtatttaagtctttgggtaaacctggatccatgcatttaagacctattgctactccttaccctgttaaggaatggttgcaaccaaaacgttataaggcacacttgatgggtaccacatatgtctatgacttcccagaattattccgccaagcatcgtcatcccaatggaaaaatttctctgcagatgttaagttaacagatgatttctttatttccaacgagttgattgaagatgaaaacggcgaattaactgaggtggaaagagaacctggtgccaacgctattggtatggttgcctttaagattactgtaaagactcctgaatatccaagaggccgtcaatttgttgttgttgctaacgatatcacattcaagatcggttcctttggtccacaagaagacgaattcttcaataaggttactgaatatgctagaaagcgtggtatcccaagaatttacttggctgcaaactcaggtgccagaattggtatggctgaagagattgttccactatttcaagttgcatggaatgatgctgccaatccggacaagggcttccaatacttatacttaacaagtgaaggtatggaaactttaaagaaatttgacaaagaaaattctgttctcactgaacgtactgttataaacggtgaagaaagatttgtcatcaagacaattattggttctgaagatgggttaggtgtcgaatgtctacgtggatctggtttaattgctggtgcaacgtcaagggcttaccacgatatcttcactatcaccttagtcacttgtagatccgtcggtatcggtgcttatttggttcgtttgggtcaaagagctattcaggtcgaaggccagccaattattttaactggtgctcctgcaatcaacaaaatgctgggtagagaagtttatacttctaacttacaattgggtggtactcaaatcatgtataacaacggtgtttcacatttgactgctgttgacgatttagctggtgtagagaagattgttgaatggatgtcttatgttccagccaagcgtaatatgccagttcctatcttggaaactaaagacacatgggatagaccagttgatttcactccaactaatgatgaaacttacgatgtaagatggatgattgaaggtcgtgagactgaaagtggatttgaatatggtttgtttgataaagggtctttctttgaaactttgtcaggatgggccaaaggtgttgtcgttggtagagcccgtcttggtggtattccactgggtgttattggtgttgaaacaagaactgtcgagaacttgattcctgctgatccagctaatccaaatagtgctgaaacattaattcaagaacctggtcaagtttggcatccaaactccgccttcaagactgctcaagctatcaatgactttaacaacggtgaacaattgccaatgatgattttggccaactggagaggtttctctggtggtcaacgtgatatgttcaacgaagtcttgaagtatggttcgtttattgttgacgcattggtggattacaaacaaccaattattatctatatcccacctaccggtgaactaagaggtggttcatgggttgttgtcgatccaactatcaacgctgaccaaatggaaatgtatgccgacgtcaacgctagagctggtgttttggaaccacaaggtatggttggtatcaagttccgtagagaaaaattgctggacaccatgaacagattggatgacaagtacagagaattgagatctcaattatccaacaagagtttggctccagaagtacatcagcaaatatccaagcaattagctgatcgtgagagagaactattgccaatttacggacaaatcagtcttcaatttgctgatttgcacgataggtcttcacgtatggtggccaagggtgttatttctaaggaactggaatggaccgaggcacgtcgtttcttcttctggagattgagaagaagattgaacgaagaatatttgattaaaaggttgagccatcaggtaggcgaagcatcaagattagaaaagatcgcaagaattagatcgtggtaccctgcttcagtggaccatgaagatgataggcaagtcgcaacatggattgaagaaaactacaaaactttggacgataaactaaagggtttgaaattagagtcattcgctcaagacttagctaaaaagatcagaagcgaccatgacaatgctattgatggattatctgaagttatcaagatgttatctaccgatgataaagaaaaattgttgaagactttgaaa

SEQ ID NO: 4 - DNA, 1206 bp, synthetic construct
  source 1..1206: mol_type = other DNA
atgggttcttactcttctgacgatgtcgaagtcatcagagaagctggtagagctcaaggtttagctaccattcttgctattggtaccgccactccaccaaactgtgttgcccaagctgactacgctgactactacttcagagtcaccaagtctgaacacatggtcgatttgaaggaaaaattcaagagaatctgtgaaaagaccgctatcaagaagcgttacttggctctaacagaagattatttgcaagaaaatccaactatgtgtgaattcatggctccatccttgaacgctagacaagatttggtcgttaccggtgtcccaatgctaggtaaggaagctgccgttaaggccattgacgaatggggtttgccaaagtccaagatcacccacttgatcttctgtaccaccgcaggtgtcgacatgccaggtgccgattaccaattggttaaattgttgggtttgagtccatctgtcaagagatacatgttgtaccaacaaggttgtgctgccggtggtactgttctaagattggccaaggacttggccgaaaacaacaagggttccagagttttgattgtctgtagcgaaatcactgctatcttatttcacggtccaaacgaaaaccatttggattcattggtcgctcaagctttgttcggtgacggtgctgccgccttgattgttggttctggacctcacttggctgtcgaaagacctatcttcgaaattgtttccactgatcaaactattttgccagacactgaaaaggctatgaagttgcatttacgtgaaggtggtttgactttccaattgcacagagatgttccattgatggttgctaagaacattgaaaatgctgccgaaaaagctttgtccccattgggtatcaccgactggaactctgttttctggatggttcacccaggtggtagagctatcttagaccaagttgaacggaagttgaacttaaaggaggacaagttacgtgcttctagacacgtcctttctgaatacggtaacttgatttctgcttgcgtcttgttcattatcgatgaagttagaaagagatccatggctgaaggtaaatccaccactggtgaaggtttggactgtggtgttttgtttggcttcggaccaggtatgactgttgaaaccgttgtcttgagatctgtcagagttactgctgctgttgctaacggtaac

SEQ ID NO: 5 - DNA, 1116 bp, synthetic construct
  source 1..1116: mol_type = other DNA
atgagtgaatctccaatgttcgctgccaacggcatgccaaaggtaaatcaaggtgctgaagaagatgtcagaattttaggttacgacccattagcttctccagctctccttcaagtgcaaatcccagccacaccaacttctttggaaactgccaagagaggtagaagagaagctatagatattattaccggtaaagacgacagagttcttgtcattgtcggtccttgttccatccatgatctagaagccgctcaagaatacgctttgagattaaagaaattgtcagatgaattaaaaggtgatttatccatcattatgagagcatacttggagaagccaagaacaaccgtcggctggaaaggtctaattaatgaccctgatgttaacaacactttcaacatcaacaagggtttgcaatccgctagacaactgtttgtcaacttgacaaatatcggtttgccaattggttctgaaatgcttgataccatttctcctcaatacttggctgatttggtctccttcggtgccattggtgccagaaccaccgaatctcaactgcacagagaattggcctccggtttgtctttcccagttggtttcaagaacggtaccgatggtaccttaaatgttgctgtggatgcttgtcaagccgctgctcattctcaccatttcatgggtgttactttgcatggtgttgctgctatcaccactactaagggtaacgaacactgcttcgttattctaagaggtggtaaaaagggtaccaactacgacgctaagtccgttgcagaagctaaggctcaattgcctgccggttccaacggtctaatgattgactactctcacggtaactccaataaggatttcagaaaccaaccaaaggtcaatgacgttgtttgtgagcaaatcgctaacggtgaaaacgccattaccggtgtcatgattgaatcaaacatcaacgaaggtaaccaaggcatcccagccgaaggtaaagccggcttgaaatatggtgtttccatcactgatgcttgtataggttgggaaactactgaagacgtcttgaggaaattggctgctgctgtcagacaaagaagagaagttaacaagaaaggatcc

SEQ ID NO: 6 - DNA, 768 bp, synthetic construct
  source 1..768: mol_type = other DNA
atggatttcacaaaaccagaaactgttttaaatctacaaaatattagagatgaattagttagaatggaggattcgatcatcttcaaatttattgagaggtcgcatttcgccacatgtccttcagtttatgaggcaaaccatccaggtttagaaattccgaattttaaaggatctttcttggattgggctctttcaaatcttgaaattgcgcattctcgcatcagaagattcgaatcacctgatgaaactcccttctttcctgacaagattcagaaatcattcttaccgagcattaactacccacaaattttggcgccttatgccccagaagttaattacaatgataaaataaaaaaagtttatattgaaaagattataccattaatttcgaaaagagatggtgatgataagaataacttcagttctgttgccactagagatatagaatgtttgcaaagcttgagtaggagaatccactttggcaagtttgttgctgaagccaagttccaatcggatatcccgctatacacaaagctgatcaaaagtaaagatgtcgaggggataatgaagaatatcaccaattctgccgttgaagaaaagattctagaaagattaactaagaaggctgaagtctatggtgtggaccctaccaacgagtcaggtgaaagaaggattactccagaatatttggtaaaaatttataaggaaattgttatacctatcactaaggaagttgaggtggaatacttgctaagaaggttggaagag

SEQ ID NO: 7 - DNA, 1119 bp, synthetic construct
  source 1..1119: mol_type = other DNA
atggttgctgaattgaccgcattacgcgatcaaattgatgaagtcgataaagcgctgctgaatttattagcgaagcgtctggaactggttgctgaagtgggcgaggtgaaaagccgctttggactgcctatttatgttccggagcgcgaggcatctatcttggcctcgcggcgcgcagaggcggaagctctgggtgtaccgccagatctgattgaggatgttttgcgtcgggtgatgcgtgaatcttactccagtgaaaacgacaaaggatttaaaacactttgtccgtcactgcgtccggtggttatcgtcggcggtggcggtcagatgggacgcctgttcgagaagatgctgacactatcgggttatcaggtgcggattctggagcaacatgactgggatcgagcggctgatattgttgccgatgccggaatggtgattgttagtgtgccaatccacgttactgagcaagttatcggcaaattaccgcctttaccgaaagattgtattctggttgatctggcatcagtgaaaaatggaccattacaggccatgctggcggcgcacgatggcccggtactggggttacacccgatgttcggcccggacagcggtagcctggcaaagcaagttgtggtctggtgtgatggacgtaagccggaagcataccaatggtttctggagcaaattcaggtctggggcgctcggctgcatcgtattagcgctgtcgagcacgatcagaatatggcgtttattcaggctctgcgccactttgctacttttgcttatgggctgcatctggcagaagaaaatgttcagcttgagcaacttctggcgctctcttcgccgatttaccgccttgagctggcgatggtcgggcgactgtttgctcaggatccgcagctttatgccgacattattatgtcgtcagagcgtaatctggcgttaatcaaacgttactataagcgtttcggcgaggcgattgagttgctggagcagggcgataagcaggcgtttattgacagtttccgcaaggtggagcactggttcggcgattacgttcagcgttttcagagtgaaagccgcgtgttattgcgtcaggcgaatgacaaccgccag

SEQ ID NO: 8 - DNA, 1560 bp, synthetic construct
  source 1..1560: mol_type = other DNA
atgaagccagaagatttcagagcttccgctaccagaccattcaccggtgaagaatacttagcttctttgagagatgacagagaaatctacatttatggtgatagagttaaagatgttacctctcacccagcttttagaaacgcagccgcttccatggctagattatacgatgccttgcacgacccacaatccaaggaaaagttgtgttgggaaactgacactgggaatggtggttacactcacaagttcttcagatacgccagatctgctgacgaactgagacaacaaagagatgctatcgctgaatggtctagattaacctacggttggatgggtagaacaccagactacaaggctgctttcggctctgccttgggtgctaacccaggtttctacggtagattcgaagacaatgctaagacctggtacaagagaatccaggaagcttgtttgtatttgaaccatgccattgttaacccacctattgaccgtgacaaaccagttgaccaagttaaggacgttttcatctctgtcgatgaagaagttgatggtggcatcgttgtctccggtgccaaggttgtcgctaccaactctgctttgacccactacaacttcgtcggtcaaggttcggctcaattgctaggtgacaacaccgactttgctttgatgttcatcgctccaatgaacactccaggtatgaaactaatctgtagaccatcttacgagttagtcgccggtattgctggttctccatttgactacccattatcctccagattcgatgaaaacgacgctattttggttatggacaaggtcttcattccatgggaaaacgtcttgatttacagggatttcgaaagatgtaagcaatggttcccacaaggtggtttcggtcgtttatttccaatgcaaggttgtactagattggctgttaagctcgactttatcactggtgctttgtacaaagctttgcaatgcactggttctttggaattcagaggtgttcaagctcaagttggtgaagtcgtcgcttggagaaacttgttctggtcattaaccgatgctatgtacggtaacgcctctgaatggcacggtggtgctttcttgccgtccgctgaagccttgcaagcttacagagtcttggccccacaagcttacccagaaattaagaagactattgaacaagtcgttgcctctggtttgatctacttgccatctggtgtccgtgatttgcacaaccctcaattggataagtacttgtctacttactgtcgtggttccggtggtatgggtcacagagaacgtatcaagatcttgaagttgttgtgggatgctatcggttccgaattcggtggtagacacgaattgtacgaaatcaactatgctggttctcaagatgaaatcagaatgcaagccctaagacaagctattggtagcggtgccatgaagggtatgttgggtatggttgaacaatgtatgggtgactacgacgaaaacggttggactgttccacatttgcataacccagatgacattaacgttttggacagaattagacaa

SEQ ID NO: 9 - DNA, 510 bp, synthetic construct
  source 1..510: mol_type = other DNA
atgcaattggacgaacaaagattgagattcagagatgctatggcttctttgtccgccgctgttaacatcatcaccactgaaggtgacgccggtcaatgtggtatcactgctaccgctgtctgctccgttaccgacactccaccatctttaatggtttgtatcaacgctaactctgctatgaacccagttttccaaggtaacggtaagttgtgtgtcaatgttttgaaccacgaacaagaattgatggctagacactttgctggtatgactggtatggccatggaagaacgtttcagcttgtcttgttggcaaaagggtccattggctcaaccagtcttgaaaggctctctagcctccttagaaggtgaaatcagagatgtccaagctattggtacccatttggtctacttggttgaaatcaagaacattattttatcagctgaaggtcacggtttgatttacttcaagagaagattccaccctgttatgttggaaatggaagctgccatt

SEQ ID NO: 10 - DNA, 1065 bp, synthetic construct
  source 1..1065: mol_type = other DNA
atgccaagattgtgtaaacctgctgtttctgctcctgaatatactattactatggaagaaactttggagttcgctaaacaagcacatgctggtaaaccacaattgccattggctttgagattgattagaaatactggtgttttgaaaagacatattgttcaaccaattgaaaaaactttgggtcatcctggtttgactgaaagaaatttgatttatgaagctgaatctaaaaaaatgtgtccacctgttattgaagaagctttgcaaaatgctgatatgactgctagagatattgatgctattatttatgtttcttgtactggttttttgatgccatctttgactgcttggttgattaataaaatgggttttagatctgatactagacaaattccaattgctcaattgggttgtgctgctggtggtgctgctgttaatagagctcatgatttttgtttggctcatcctggttctaatgttttgattgttgcttgtgaattgtgttctttgtgttatcaaccaactgctgatgatattggttctttgttgtctgatggtttgtttggtgatgctgttgctgctgctgttgttagaggtaatggtggtgtgggtattgaagttgaaagaaatgcttcttatttaattccgaatacggaagaatggatttcttattctgttagagatactggttttcattttcaattggatagaagagttcctggtactatggaaccattggctcctgttttgagagaatttgctaaggaccattcttgggatgctggtaaattggatttttatattgttcatgctggtggtccaagaattttggatgatttggctagatttttggatgttgatagacaagtttttagacattcttggtctactttgactgaatatggtaatattgcttctgctgttgtttttgatgctgctagaagattgtttgaagaaggttctgctaaacctgatgctactggtatgattgctggttttggtcctggtattactgctgaaatggctttgggtacttggggtactgatggtactggtacttctaat

SEQ ID NO: 11 - DNA, 27 bp, synthetic construct
  source 1..27: mol_type = other DNA
cccgctgctaaaagagttaagttggat

SEQ ID NO: 12 - DNA, 57 bp, synthetic construct
  source 1..57: mol_type = other DNA
aagagaacagctgacggttctgaatttgagtctcctaaaaagaaaagaaaagtcgag

SEQ ID NO: 13 - DNA, 96 bp, synthetic construct
  source 1..96: mol_type = other DNA
acaaataaaagaaaaagagagcaaatttctactgataatgaagcaaaaatgcaaattcaagaggagaaatcacctaaaaagaaaaggaaaaaaagg

SEQ ID NO: 14 - DNA, 680 bp, synthetic construct
  source 1..680: mol_type = other DNA
cagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaa

SEQ ID NO: 15 - DNA, 225 bp, synthetic construct
  source 1..225: mol_type = other DNA
agcttttgattaagccttctagtccaaaaaacacgtttttttgtcatttatttcattttcttagaatagtttagtttattcattttatagtcacgaatgttttatgattctatatagggttgcaaacaagcatttttcattttatgttaaaacaatttcaggtttaccttttattctgcttgtggtgacgcgtgtatccgcccgctcttttggtcacccatgtat

SEQ ID NO: 16 - DNA, 700 bp, synthetic construct
  source 1..700: mol_type = other DNA
cacccatgaaccacacggttagtccaaaaggggcagttcagattccagatgcgggaattagcttgctgccaccctcacctcactaacgctgcggtgtgcggatacttcatgctatttatagacgcgcgtgtcggaatcagcacgcgcaagaaccaaatgggaaaatcggaatgggtccagaactgctttgagtgctggctattggcgtctgatttccgttttgggaatcctttgccgcgcgcccctctcaaaactccgcacaagtcccagaaagcgggaaagaaataaaacgccaccaaaaaaaaaaaaataaaagccaatcctcgaagcgtgggtggtaggccctggattatcccgtacaagtatttctcaggagtaaaaaaaccgtttgttttggaatttcccatttcgcggccacctacgccgctatctttgcaacaactatctgcgataactcagcaaattttgcatattcgtgttgcagtattgcgataatgggagtcttacttccaacataacggcagaaagaaatgtgagaaaattttgcatcctttgcctccgttcaagtatataaagtcggcatgcttgataatctttctttccatcctacattgttctaattattcttattctcctttattctttcctaacataccaagaaattaatcttctgtcattcgcttaaacactatatcaataa

SEQ ID NO: 17 - DNA, 225 bp, synthetic construct
  source 1..225: mol_type = other DNA
gccaattggtgcggcaattgataataacgaaaatgtcttttaatgatctgggtataatgaggaattttccgaacgtttttactttatatatatatatacatgtaacatatattctatacgctatagagaaaggaaatttttcaattaaaaaaaaaatagagaaagagtttcacttcttgattatcgctaacactaatggttgaagtactgctactttaattttat

SEQ ID NO: 18 - DNA, 700 bp, synthetic construct
  source 1..700: mol_type = other DNA
gtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcatcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaaca

SEQ ID NO: 19 - DNA, 225 bp, synthetic construct
  source 1..225: mol_type = other DNA
gcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttc

SEQ ID NO: 20 - DNA, 676 bp, synthetic construct
  source 1..676: mol_type = other DNA
tgtggagtgtttgcttggattctttagtaaaaggggaagaacagttggaagggccaaagtggaagtcacaaaacagtggtcctatataaaagaacaagaaaaagattatttatatacaactgcggtcacaagaagcaacgcgagagagcacaacacgctgttatcacgcaaactatgttttgacaccgagccatagccgtgattgtgcgtcacattgggcgataatgaacgctaaatgaccaactcccatccgtaggagccccttagggcgtgccaatagtttcacgcgcttaatgcgaagtgctcggaacggacaactgtggtcgtttggcaccgggaaagtggtactagaccgagagtttcgcatttgtatggcaggacgttctgggagcttcgcgtctaaagctttttcgggcgcgaaatgcagaccagaccagaacaaaacaactgacaagaaggcgtttaatttaatatgttgttcactcgcgcctgggctgttgttattcggctagatacatacgtgtttgtgcgtatgtagttatatcatatataagtatattaggatgaggcggtgaaagagattttttttttttcgcttaatttattcttttctctatcttttttcctacatcttgttcaaaagagtagcaaaaacaacaatcaatacaataaaata

SEQ ID NO: 21 - DNA, 225 bp, synthetic construct
  source 1..225: mol_type = other DNA
attgaattgaattgaaatcgatagatcaatttttttcttttctctttccccatcctttacgctaaaataatagtttattttattttttgaatattttttatttatatacgtatatatagactattatttatcttttaatgattattaagatttttattaaaaaaaaattcgctcctcttttaatgcctttatgcagtttttttttcccattcgatatttctatgt

SEQ ID NO: 22 - DNA, 700 bp, synthetic construct
  source 1..700: mol_type = other DNA
ccttgccaacagggagttcttcagagacatggaggctcaaaacgaaattattgacagcctagacatcaatagtcatacaacagaaagcgaccacccaactttggctgataatagcgtataaacaatgcatactttgtacgttcaaaatacaatgcagtagatatatttatgcatattacatataatacatatcacataggaagcaacaggcgcgttggacttttaattttcgaggaccgcgaatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagacaccgcctcgtttctttttcttcgtcgaaaaaggcaataaaaatttttatcacgtttctttttcttgaaaatttttttttttgatttttttctctttcgatgacctcccattgatatttaagttaataaacggtcatcaatttctcaagtttcagtttcatttttcttgttctattacaactttttttacttcttgctcattagaaagaaagcatagcaatctaatctaagttttaattacaaa

SEQ ID NO: 23 - DNA, 225 bp, synthetic construct
  source 1..225: mol_type = other DNA
agtgcttttaactaagaattattagtcttttctgcttattttttcatcatagtttagaacactttatattaacgaatagtttatgaatctatttaggtttaaaaattgatacagttttataagttactttttcaaagactcgtgctgtctattgcataatgcactggaaggggaaaaaaaaggtgcacacgcgtggctttttcttgaatttgcagtttgaaaaat

SEQ ID NO: 24 - DNA, 536 bp, synthetic construct
  source 1..536: mol_type = other DNA
taactcttcgtatgaggattttcgatggagcaggatgaggagaaatagtaccacatgtatatatccattacaaaaaggtttatatacaattacaatagacccttgttggggtttctgaaaaaagaagtagtcgatgccatcggcaataatacggaattacgagaaacacaatcccgatccttttttgggtaattacttcaccgattctaccgatttatcatgccaaaaaaaattcaccgtgggttctagaagtgccctttgaggattgtagccactctaacccacacggcctccttactagctgactaaggtgacaaaaccgcaaggactggaaagtcgccactcatctgaaaattctcaagtttttcactactgagtttatgctttcgaatttttttgttcggtaatagcacggcggttcgattcaattccgccgctccgagcgatgctccgcaaaactcagtaataagctttctgatggttcaccccttttttagcacgcggggtgtaactcaacagaaaaatgtgccatagaa

SEQ ID NO: 25 - DNA, 764 bp, synthetic construct
  source 1..764: mol_type = other DNA
tcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgt

SEQ ID NO: 26 - DNA, 483 bp, synthetic construct
  source 1..483: mol_type = other DNA
ccacaagtaaagctcgttgaccagttgatcagttgaggggggtacacacgactagcgctttcagatattaaaaagtttagatgtaggttttagcggtaacagttatataaatcgtgtttcttctcttgatgaaacaaaaaaatgctagaaaaactttgtcgtttcttacttttggtgcgctttgcagttttcgtggctagacttagaatcatttctcctcagatttcttgattaaagtttggtgcgaagccctactctaacattggtgttcttcttttcattcacgcaagttaagtccaggaaggtgagcaaatgctcatccttctgttcatgcgtgacggctgaattatccttatctggcgtacccgtgcagccgtttccgtgcctcggttcctccgagatatccttagggaccgccagggaccatgattgcgtcaactgttgtcaccgctccagaggatcctctgtaaccttttcaaccat

SEQ ID NO: 27 - DNA, 1610 bp, synthetic construct
  source 1..1610: mol_type = other DNA
agcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctccagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaaaatgggtaaaaagcctgaactcaccgcgacgtctgtcgagaagtttctgatcgaaaagttcgacagcgtgtccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaatttagcgagagcctgacctattgcatctcccgccgtgcacagggtgtcacgttgcaagacctgcctgaaaccgaactgcccgctgttctgcaaccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataaagtaactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactg

SEQ ID NO: 28 - DNA, 200 bp, synthetic construct
  source 1..200: mol_type = other DNA
accaatcagtaaaaatcaacggttaacgacattactatatatataatataggaagcatttaatagaacagcatcgtaatatatgtgtactttgcagttatgacgccagatggcagtagtggaagatattctttattgaaaaatagcttgtcaccttacgtacaatcttgatccggagcttttctttttttgccgattaag

SEQ ID NO: 29 - DNA, 269 bp, synthetic construct
  source 1..269: mol_type = other DNA
caggaaaatatacatcgcagggggttgacttttaccatttcaccgcaatggaatcaaacttgttgaagagaatgttcacaggcgcatacgctacaatgacccgattcttgctagccttttctcggtcttgcaaacaaccgccggcagcttagtatataaatacacatgtacatacctctctccgtatcctcgtaatcattttcttgtatttatcgtcttttcgctgtaaaaactttatcacacttatctcaaatacacttattaaccgc

SEQ ID NO: 30 - DNA, 856 bp, synthetic construct
  source 1..856: mol_type = other DNA
aattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctgcaggccttttgaaaagcaagcataaaagatc

(19) United States
(12) Patent Application Publication
(10) Pub. No.: US 2026/0125689 A1
(43) Pub. Date: May 7, 2026

(54) Novel Nuclear Localization Sequence Mutant and Method for Improving Biosynthetic Efficiency Using Same

(71) Applicant: Jiangnan University, Wuxi (CN)

(72) Inventors: Ye LI, Wuxi (CN); Zhonghu BAI, Wuxi (CN); Zhenhao FU, Wuxi (CN); Pingxin LIN, Wuxi (CN)

(21) Appl. No.: 19/426,208

(22) Filed: Dec. 19, 2025

(30) Foreign Application Priority Data
Jun. 24, 2025 (CN) 2025108527270

(51) Int. Cl. C12N 15/52; C12N 9/00; C12N 9/02; C12N 9/06; C12N 9/10; C12N 9/90; C12P 7/22; C12P 17/06

(52) U.S. Cl. CPC C12N 15/52; C12N 9/001; C12N 9/0028; C12N 9/0071; C12N 9/1029; C12N 9/1085; C12N 9/90; C12N 9/93; C12P 7/22; C12P 17/06; C12Y 103/01012; C12Y 114/14009; C12Y 205/01054; C12Y 504/99005; C12Y 604/01002

(57) ABSTRACT

Disclosed are a novel nuclear localization sequence (NLS) mutant and a method for improving biosynthetic efficiency using same, belonging to the field of bioengineering. According to the present disclosure, a novel NLS is screened out, and recombinant yeast with improved metabolite synthesis efficiency is constructed by applying the NLS obtained through screening, which provides key technical support for developing a high-yield, stable and cost-effective yeast biosynthesis platform, and facilitates the use of recombinant yeast as a cell factory for large-scale industrial biomanufacturing.

### REFERENCE TO SEQUENCE LISTING

The instant application contains a Sequence Listing in XML format as a file named “YGHY-2025-22-SEQ.xml”, created on Dec. 11, 2025, of 50576 Bytes in size, and which is hereby incorporated by reference in its entirety.

### TECHNICAL FIELD

The present disclosure relates to a novel nuclear localization sequence (NLS) mutant and a method for improving biosynthetic efficiency using same, belonging to the field of bioengineering.

### BACKGROUND

With the rapid development of synthetic biology, producing valuable compounds by engineering microbial cell factories through metabolic engineering as an alternative to conventional production methods has emerged as a more environmentally friendly and sustainable solution. However, designing functional metabolic pathways faces multiple challenges, including controlling the expression intensity of enzymes, or altering the product tendency of key enzymes, suboptimal physicochemical reaction environments, intermediate loss caused by endogenous competing pathways, and metabolite toxicity. Among eukaryotic microorganisms, Saccharomyces cerevisiae grows vigorously at low pH, is simple in nutritional requirements, is not susceptible to viral contamination, and has high tolerance to substrate and product toxicity. Additionally, S. cerevisiae possesses a complete whole genome sequence, is detailed in gene annotation resources and simple in gene manipulation methods, facilitates post-translational modification, and can express complex heterologous enzymes, making it favored for industrial applications.

Although most current strategies for optimizing metabolic pathways, including protein fusion and synthesis of protein scaffolds, can enhance the productivity of certain metabolic pathways, as metabolic pathways become increasingly complex, the number of enzymes that can be built into scaffolds without competition is limited by available binding domains, and both protein fusion and scaffolds will exert adverse effects on enzyme activity. Leveraging the functional characteristics of various organelles to address these obstacles to metabolic flux has emerged as a new focus in metabolic engineering modification of S. cerevisiae, which involves compartmentalizing metabolic pathways within subcellular organelles. The unique physicochemical environment (e.g., pH and redox potential), enzymes, metabolites, and cofactors in each organelle provide favorable conditions for different metabolic pathways. By confining pathways within smaller subcellular compartments, the local concentrations of substrates and enzymes can be increased, thereby accelerating reaction rates and improving productivity. Restricting intermediates within organelles may also inhibit their conversion to byproducts and reduce their toxic effects on cellular processes.

Current organelle engineering mainly focuses on organelles such as peroxisomes, mitochondria, endoplasmic reticulum, and lipid droplets. Numerous studies have demonstrated that pathway compartmentalization in yeast organelles can yield significant improvement compared to cytoplasmic pathways, but this improvement is only applicable to certain specific pathways and lacks strong universality, with the nuclear compartment remains underexplored to date. Due to unfavorable physiological environments and insufficient supply of essential cofactors or precursors, enzyme activity may be reduced. Therefore, the selection of target organelles for metabolic pathways is crucial, and the development of novel organelles offers new opportunities to increase the yield of metabolic pathways.

### SUMMARY

The present disclosure provides a novel yeast NLS mutant and a method for improving biosynthetic efficiency using a yeast NLS. According to the method, metabolic pathways for target products are first constructed, an NLS is then added to proteins related to the metabolic pathways, and finally, the target products are produced by fermentation.

The present disclosure provides the following technical solutions to achieve the above objective:

The present disclosure provides a novel yeast NLS, a nucleotide sequence of the novel yeast NLS is as set forth in SEQ ID NO. 1.

The present disclosure further provides a nucleic acid construct including the yeast NLS as set forth in SEQ ID NO. 1.

In one embodiment, the nucleic acid construct includes at least one NLS and at least one gene related to a biological metabolic pathway; and the NLS is linked upstream or downstream of the gene related to the biological metabolic pathway for guiding a directional localization of a gene expression product to a host cell nucleus.

In one embodiment, a protein encoded by the gene related to the biological metabolic pathway is involved in a synthetic pathway of a target metabolite in a microorganism.

In one embodiment, the genes related to the biological metabolic pathways include but are not limited to: 2-pyrone synthase gene (Gh2-PS), acetyl-CoA carboxylase gene (Acc1^{mut}), 3-deoxy-D-arabino-heptulosonate-7-phosphate synthase gene (ARO4), chorismate mutase gene (ARO7), prephenate dehydrogenase gene (TyrA), 4-hydroxyphenylacetate 3-monooxygenase gene (PaHpaB), flavin reductase gene (EcHpaC), and 1,3,6,8-tetrahydroxynaphthalene synthase gene (RppA).

In one embodiment, the host is a eukaryotic microorganism, including but not limited to S. cerevisiae.

The present disclosure further provides recombinant S. cerevisiae, and the recombinant S. cerevisiae includes a gene localized under the guidance of the yeast NLS.

In one embodiment, the recombinant S. cerevisiae includes an Acc1 gene with a nucleotide sequence as set forth in SEQ ID NO. 3 and a Gh2-PS gene with a nucleotide sequence as set forth in SEQ ID NO. 4, and 3′ ends of the Acc1 gene and the Gh2-PS gene are separately linked to the NLS SV4040 as set forth in SEQ ID NO. 1.

In one embodiment, the recombinant S. cerevisiae includes the Acc1 gene with the nucleotide sequence as set forth in SEQ ID NO. 3 and an RppA gene with a nucleotide sequence as set forth in SEQ ID NO. 10, and 3′ ends of the Acc1 gene and the RppA gene are separately linked to the NLS SV4040 as set forth in SEQ ID NO. 1.

In one embodiment, the recombinant S. cerevisiae includes an ARO4 gene with a nucleotide sequence as set forth in SEQ ID NO. 5, an ARO7 gene with a nucleotide sequence as set forth in SEQ ID NO. 6, a TyrA gene with a nucleotide sequence as set forth in SEQ ID NO. 7, a PaHpaB gene with a nucleotide sequence as set forth in SEQ ID NO. 8 and an EcHpaC gene with a nucleotide sequence as set forth in SEQ ID NO. 9, and 3′ ends of the ARO4 gene, the ARO7 gene, the TyrA gene, the PaHpaB gene and the EcHpaC gene are separately linked to the NLS SV4040 as set forth in SEQ ID NO. 1.

The present disclosure further provides a method for improving biosynthetic efficiency, including following steps:
(a) constructing a recombinant nucleic acid construct, where the construct includes: at least one NLS, and at least one gene encoding a key enzyme in a synthetic pathway of a target metabolite; and the NLS is operably linked upstream or downstream of the gene;
(b) introducing the recombinant nucleic acid construct into a eukaryotic microbial host cell;
(c) culturing the host cell under suitable conditions to enable the expression and localization of the key enzyme within a cell nucleus; and
(d) collecting the target metabolite.

In one embodiment, the target metabolite includes but are not limited to products of the metabolic pathways of triacetic acid lactone (TAL), mevalonic acid (MVA), tryptophol, methyl anthranilate (Me-AA), 2-phenylethanol (2-PE), tyrosol, or hydroxytyrosol (HT).

In one embodiment, the expression in step (3) includes integrated expression and episomal expression.

In one embodiment, the NLS includes SV4040 with a nucleotide sequence as set forth in SEQ ID NO. 1, SV40 with a nucleotide sequence as set forth in SEQ ID NO. 2, or cMyc as set forth in SEQ ID NO. 11.

The present disclosure further provides application of the yeast NLS, the recombinant microbial cells, or the method in the field of fermentation.

In one embodiment, the application includes preparation of food, medicine or health care products containing one or more substances selected from hydroxytyrosol, triacetic acid lactone, mevalonic acid and tryptophol.

### Beneficial Effects

(1) The present disclosure has screened the novel NLS, which significantly improves the biosynthetic efficiency compared with the existing NLS, thereby increasing the yield of triacetic acid lactone-producing S. cerevisiae engineered strain TAL (SV4040) whose key enzyme is localized and expressed in the nucleus by 26.7%, increasing the yield of flaviolin-producing S. cerevisiae engineered strain Flaviolin (SV4040) whose key enzyme is localized and expressed in the nucleus by 39%, and increasing the yield of hydroxytyrosol-producing S. cerevisiae engineered strain HT (SV4040) whose key enzyme is localized and expressed in the nucleus by 103%.
(2) The present disclosure applies the NLS obtained through screening to construct recombinant yeast with improved metabolite synthesis efficiency, which provides key technical support for developing a high-yield, stable and cost-effective yeast biosynthesis platform, and facilitates the use of recombinant yeast as a cell factory for large-scale industrial biomanufacturing.

### BRIEF DESCRIPTION OF FIGURES

FIG. 1 is a schematic diagram of a TAL cytoplasmic pathway expression cassette.

FIG. 2 shows the qualitative analysis of TAL, including high-performance liquid chromatography (HPLC) detection results of a TAL fermentation broth and liquid chromatography-mass spectrometry (LC-MS) detection results of the TAL fermentation broth.

FIG. 3 is a schematic diagram of a TAL nuclear pathway expression cassette.

FIG. 4 shows the fermentation results of TAL engineered strain.

FIG. 5 is a schematic diagram of a flaviolin cytoplasmic pathway expression cassette.

FIG. 6 is a schematic diagram of a flaviolin nuclear pathway expression cassette.

FIG. 7 shows the fermentation results of flaviolin engineered strain.

FIG. 8 shows the fermentation results of flaviolin cytoplasmic pathway engineered strain Flaviolin (Cytosol), as well as nuclear pathway engineered strains Flaviolin (BPSV40) and Flaviolin (HEH2).

FIG. 9 is a schematic diagram of a hydroxytyrosol cytoplasmic pathway expression cassette.

FIG. 10A shows HPLC detection results of an HT fermentation broth;

FIG. 10B shows LC-MS detection results of the HT fermentation broth.

FIG. 11 is a schematic diagram of an HT nuclear pathway expression cassette.

FIG. 12 shows the fermentation results of HT engineered strain.

### DETAILED DESCRIPTION

### (I) Culture Media

Selective SC medium: 6.7 g/L yeast nitrogen base (YNB), 1.27 g/L amino acid dropout mix, and 20 g/L glucose.

YTD medium: 10 g/L yeast extract, 20 g/L peptone, and 20 g/L glucose.

### (II) Detection Method

Detection method of TAL: At the end of fermentation, a culture was centrifuged at 12,000 rpm for 10 min. The supernatant was filtered through a 0.22 μm filter membrane, and 10 μL of a sample was injected into a HPLC system. Metabolites were separated using a Shimadzu Shim-pack GIST C18 chromatographic column (5 μm, 150 mm×4.6 mm). A column oven was maintained at 30° C. Gradient elution was performed on the sample using two solvents: an H₂O solution (Solvent A) containing 1% acetic acid and a CH₃CN solution (Solvent B) containing 1% acetic acid. The gradient program started with the linearly changing solvent B from 2% to 7% (0-5.0 min), from 7% to 95% (5.0-7.0 min), holding at 95% (7.0-9.0 min), from 95% to 2% (9.0-11.0 min), and holding at 2% (11.0-20.0 min). The flow rate was set at 1 mL·min⁻¹, and the detection wavelength was 280 nm. The TAL concentration was quantified based on a standard curve of TAL standards.

Detection method of HT: HPLC was used for detection. A fermentation broth was centrifuged to collect supernatant, which was then filtered through a microporous filter membrane. The analytical conditions were as follows: a mobile phase consisted of 80% (v/v) water containing 0.1% (w/v) formic acid and 20% (v/v) methanol at a flow rate of 1 mL·min⁻¹; separation was performed using a Shimadzu Shim-pack GIST C18 chromatographic column (5 μm, 150 mm×4.6 mm) with a column temperature maintained at 30° C.; and the detection wavelength was 280 nm; the injection volume was 10 μL.

Detection method of flaviolin: A fermentation broth was centrifuged to collect supernatant, and an absorbance value at OD₃₈₀ was detected by a microplate reader as the yield of flaviolin.

### (III) Expression Elements and Sequences

The sequences involved in the construction of expression cassettes in the specific embodiments are shown in Table 1.

TABLE 1
Sequences of gene regulatory expression elements

| Regulatory | |
|---|---|
| element | Sequence |
| pTDH3 | CAGTTCGAGTTTATCATTATCAATACTGCCATTTCAAAGAATACGTAAATAATTAATAGTAGTGATTTTCCTAA |
| | CTTTATTTAGTCAAAAAATTAGCCTTTTAATTCTGCTGTAACCCGTACATGCCCAAAATAGGGGGGGGTTACA |
| | CAGAATATATAACATCGTAGGTGTCTGGGTGAACAGTTTATTCCTGGCATCCACTAAATATAATGGAGCCCGC |
| | TTTTTAAGCTGGCATCCAGAAAAAAAAAGAATCCCAGCACCAAAATATTGTTTTCTTCACCAACCATCAGTTCA |
| | TAGGTCCATTCTCTTAGCGCAACTACAGAGAACAGGGGCACAAACAGGCAAAAAACGGGCACAACCTCAATG |
| | GAGTGATGCAACCTGCCTGGAGTAAATGATGACACAAGGCAATTGACCCACGCATGTATCTATCTCATTTTCT |
| | TACACCTTCTATTACCTTCTGCTCTCTCTGATTTGGAAAAAGCTGAAAAAAAAGGTTGAAACCAGTTCCCTGAA |
| | ATTATTCCCCTACTTGACTAATAAGTATATAAAGACGGTAGGTATTGATTGTAATTCTGTAAATCTATTTCTTAA |
| | ACTTCTTAAATTCTACTTTTATAGTTAGTCTTTTTTTTAGTTTTAAAACACCAAGAACTTAGTTTCGAATAAACAC |
| | ACATAAACAAACAAA (SEQ ID NO. 14) |
| tENO1 | AGCTTTTGATTAAGCCTTCTAGTCCAAAAAACACGTTTTTTTGTCATTTATTTCATTTTCTTAGAATAGTTTAGTT |
| | TATTCATTTTATAGTCACGAATGTTTTATGATTCTATATAGGGTTGCAAACAAGCATTTTTCATTTTATGTTAAA |
| | ACAATTTCAGGTTTACCTTTTATTCTGCTTGTGGTGACGCGTGTATCCGCCCGCTCTTTTGGTCACCCATGTAT |
| | (SEQ ID NO. 15) |
| pCCW12 | CACCCATGAACCACACGGTTAGTCCAAAAGGGGCAGTTCAGATTCCAGATGCGGGAATTAGCTTGCTGCCAC |
| | CCTCACCTCACTAACGCTGCGGTGTGCGGATACTTCATGCTATTTATAGACGCGCGTGTCGGAATCAGCACGC |
| | GCAAGAACCAAATGGGAAAATCGGAATGGGTCCAGAACTGCTTTGAGTGCTGGCTATTGGCGTCTGATTTCC |
| | GTTTTGGGAATCCTTTGCCGCGCGCCCCTCTCAAAACTCCGCACAAGTCCCAGAAAGCGGGAAAGAAATAAA |
| | ACGCCACCAAAAAAAAAAAAATAAAAGCCAATCCTCGAAGCGTGGGTGGTAGGCCCTGGATTATCCCGTACA |
| | AGTATTTCTCAGGAGTAAAAAAACCGTTTGTTTTGGAATTTCCCATTTCGCGGCCACCTACGCCGCTATCTTTG |
| | CAACAACTATCTGCGATAACTCAGCAAATTTTGCATATTCGTGTTGCAGTATTGCGATAATGGGAGTCTTACTT |
| | CCAACATAACGGCAGAAAGAAATGTGAGAAAATTTTGCATCCTTTGCCTCCGTTCAAGTATATAAAGTCGGCA |
| | TGCTTGATAATCTTTCTTTCCATCCTACATTGTTCTAATTATTCTTATTCTCCTTTATTCTTTCCTAACATACCA |
| | AGAAATTAATCTTCTGTCATTCGCTTAAACACTATATCAATAA (SEQ ID NO. 16) |
| tSSA1 | GCCAATTGGTGCGGCAATTGATAATAACGAAAATGTCTTTTAATGATCTGGGTATAATGAGGAATTTTCCGAA |
| | CGTTTTTACTTTATATATATATATACATGTAACATATATTCTATACGCTATAGAGAAAGGAAATTTTTCAATTAA |
| | AAAAAAAATAGAGAAAGAGTTTCACTTCTTGATTATCGCTAACACTAATGGTTGAAGTACTGCTACTTTAATTT |
| | TAT (SEQ ID NO. 17) |
| pPGK1 | GTGAGTAAGGAAAGAGTGAGGAACTATCGCATACCTGCATTTAAAGATGCCGATTTGGGCGCGAATCCTTTA |
| | TTTTGGCTTCACCCTCATACTATTATCAGGGCCAGAAAAAGGAAGTGTTTCCCTCCTTCTTGAATTGATGTTAC |
| | CCTCATAAAGCACGTGGCCTCTTATCGAGAAAGAAATTACCGTCGCTCGTGATTTGTTTGCAAAAAGAACAAA |
| | ACTGAAAAAACCCAGACACGCTCGACTTCCTGTCATCCTATTGATTGCAGCTTCCAATTTCGTCACACAACAAG |
| | GTCCTAGCGACGGCTCACAGGTTTTGTAACAAGCAATCGAAGGTTCTGGAATGGCGGGAAAGGGTTTAGTAC |
| | CACATGCTATGATGCCCACTGTGATCTCCAGAGCAAAGTTCGTTCGATCGTACTGTTACTCTCTCTCTTTCAAA |
| | CAGAATTGTCCGAATCGTGTGACAACAACAGCCTGTTCTCACACACTCTTTTCTTCTAACCAAGGGGGTGGTTT |
| | AGTTTAGTAGAACCTCGTGAAACTTACATTTACATATATATAAACTTGCATAAATTGGTCAATGCAAGAAATAC |
| | ATATTTGGTCTTTTCTAATTCGTAGTTTTTCAAGTTCTTAGATGCTTTCTTTTTCTCTTTTTTACAGATCATCAAG |
| | GAAGTAATTATCTACTTTTTACAACAAATATAAAACA (SEQ ID NO. 18) |
| tADH1 | GCGAATTTCTTATGATTTATGATTTTTATTATTAAATAAGTTATAAAAAAAATAAGTGTATACAAATTTTAAAGT |
| | GACTCTTAGGTTTTAAAACGAAAATTCTTATTCTTGAGTAACTCTTTCCTGTAGGTCAGGTTGCTTTCTCAGGT |
| | ATAGCATGAGGTCGCTCTTATTGACCACACCTCTACCGGCATGCCGAGCAAATGCCTGCAAATCGCTCCCCAT |
| | TTC (SEQ ID NO. 19) |
| pHHF2 | TGTGGAGTGTTTGCTTGGATTCTTTAGTAAAAGGGGAAGAACAGTTGGAAGGGCCAAAGTGGAAGTCACAA |
| | AACAGTGGTCCTATATAAAAGAACAAGAAAAAGATTATTTATATACAACTGCGGTCACAAGAAGCAACGCGA |
| | GAGAGCACAACACGCTGTTATCACGCAAACTATGTTTTGACACCGAGCCATAGCCGTGATTGTGCGTCACATT |
| | GGGCGATAATGAACGCTAAATGACCAACTCCCATCCGTAGGAGCCCCTTAGGGCGTGCCAATAGTTTCACGC |
| | GCTTAATGCGAAGTGCTCGGAACGGACAACTGTGGTCGTTTGGCACCGGGAAAGTGGTACTAGACCGAGAG |
| | TTTCGCATTTGTATGGCAGGACGTTCTGGGAGCTTCGCGTCTAAAGCTTTTTCGGGCGCGAAATGCAGACCAG |
| | ACCAGAACAAAACAACTGACAAGAAGGCGTTTAATTTAATATGTTGTTCACTCGCGCCTGGGCTGTTGTTATT |
| | CGGCTAGATACATACGTGTTTGTGCGTATGTAGTTATATCATATATAAGTATATTAGGATGAGGCGGTGAAAG |
| | AGATTTTTTTTTTTTCGCTTAATTTATTCTTTTCTCTATCTTTTTTCCTACATCTTGTTCAAAAGAGTAGCAAAA |
| | ACAACAATCAATACAATAAAATA (SEQ ID NO. 20) |
| tPGK1 | ATTGAATTGAATTGAAATCGATAGATCAATTTTTTTCTTTTCTCTTTCCCCATCCTTTACGCTAAAATAATAGTTT |
| | ATTTTATTTTTTGAATATTTTTTATTTATATACGTATATATAGACTATTATTTATCTTTTAATGATTATTAAGATT |
| | TTTATTAAAAAAAAATTCGCTCCTCTTTTAATGCCTTTATGCAGTTTTTTTTTCCCATTCGATATTTCTATGT |
| | (SEQ ID NO. 21) |
| pTEF1 | CCTTGCCAACAGGGAGTTCTTCAGAGACATGGAGGCTCAAAACGAAATTATTGACAGCCTAGACATCAATAG |
| | TCATACAACAGAAAGCGACCACCCAACTTTGGCTGATAATAGCGTATAAACAATGCATACTTTGTACGTTCAA |
| | AATACAATGCAGTAGATATATTTATGCATATTACATATAATACATATCACATAGGAAGCAACAGGCGCGTTGG |
| | ACTTTTAATTTTCGAGGACCGCGAATCCTTACATCACACCCAATCCCCCACAAGTGATCCCCCACACACCATAG |
| | CTTCAAAATGTTTCTACTCCTTTTTTACTCTTCCAGATTTTCTCGGACTCCGCGCATCGCCGTACCACTTCAAAA |
| | CACCCAAGCACAGCATACTAAATTTCCCCTCTTTCTTCCTCTAGGGTGTCGTTAATTACCCGTACTAAAGGTTT |
| | GGAAAAGAAAAAAGACACCGCCTCGTTTCTTTTTCTTCGTCGAAAAAGGCAATAAAAATTTTTATCACGTTTCT |
| | TTTTCTTGAAAATTTTTTTTTTTGATTTTTTTCTCTTTCGATGACCTCCCATTGATATTTAAGTTAATAAACGGTC |
| | ATCAATTTCTCAAGTTTCAGTTTCATTTTTCTTGTTCTATTACAACTTTTTTTACTTCTTGCTCATTAGAAAGAAA |
| | GCATAGCAATCTAATCTAAGTITTAATTACAAA (SEQ ID NO. 22) |
| tENO2 | AGTGCTTTTAACTAAGAATTATTAGTCTTTTCTGCTTATTTTTTCATCATAGTTTAGAACACTTTATATTAACGAA |
| | TAGTTTATGAATCTATTTAGGTTTAAAAATTGATACAGTTTTATAAGTTACTTTTTCAAAGACTCGTGCTGTCTA |
| | TTGCATAATGCACTGGAAGGGGAAAAAAAAGGTGCACACGCGTGGCTTTTTCTTGAATTTGCAGTTTGAAAA |
| | AT (SEQ ID NO. 23) |
| chrXI | TAACTCTTCGTATGAGGATTTTCGATGGAGCAGGATGAGGAGAAATAGTACCACATGTATATATCCATTACAA |
| 5'hom | AAAGGTTTATATACAATTACAATAGACCCTTGTTGGGGTTTCTGAAAAAAGAAGTAGTCGATGCCATCGGCAA |
| | TAATACGGAATTACGAGAAACACAATCCCGATCCTTTTTTGGGTAATTACTTCACCGATTCTACCGATTTATCA |
| | TGCCAAAAAAAATTCACCGTGGGTTCTAGAAGTGCCCTTTGAGGATTGTAGCCACTCTAACCCACACGGCCTC |
| | CTTACTAGCTGACTAAGGTGACAAAACCGCAAGGACTGGAAAGTCGCCACTCATCTGAAAATTCTCAAGTTTT |
| | TCACTACTGAGTTTATGCTTTCGAATTTTTTTGTTCGGTAATAGCACGGCGGTTCGATTCAATTCCGCCGCTCC |
| | GAGCGATGCTCCGCAAAACTCAGTAATAAGCTTTCTGATGGTTCACCCCTTTTTTAGCACGCGGGGTGTAACT |
| | CAACAGAAAAATGTGCCATAGAA (SEQ ID NO. 24) |
| ColE1 | TCATGACCAAAATCCCTTAACGTGAGTTTTCGTTCCACTGAGCGTCAGACCCCGTAGAAAAGATCAAAGGATC |
| | TTCTTGAGATCCTTTTTTTCTGCGCGTAATCTGCTGCTTGCAAACAAAAAAACCACCGCTACCAGCGGTGGTTT |
| | GTTTGCCGGATCAAGAGCTACCAACTCTTTTTCCGAAGGTAACTGGCTTCAGCAGAGCGCAGATACCAAATAC |
| | TGTTCTTCTAGTGTAGCCGTAGTTAGGCCACCACTTCAAGAACTCTGTAGCACCGCCTACATACCTCGCTCTGC |
| | TAATCCTGTTACCAGTGGCTGCTGCCAGTGGCGATAAGTCGTGTCTTACCGGGTTGGACTCAAGACGATAGTT |
| | ACCGGATAAGGCGCAGCGGTCGGGCTGAACGGGGGGTTCGTGCACACAGCCCAGCTTGGAGCGAACGACCT |
| | ACACCGAACTGAGATACCTACAGCGTGAGCTATGAGAAAGCGCCACGCTTCCCGAAGGGAGAAAGGCGGAC |
| | AGGTATCCGGTAAGCGGCAGGGTCGGAACAGGAGAGCGCACGAGGGAGCTTCCAGGGGGAAACGCCTGGT |
| | ATCTTTATAGTCCTGTCGGGTTTCGCCACCTCTGACTTGAGCGTCGATTTTTGTGATGCTCGTCAGGGGGGCG |
| | GAGCCTATGGAAAAACGCCAGCAACGCGGCCTTTTTACGGTTCCTGGCCTTTTGCTGGCCTTTTGCTCACATG |
| | TTCTTTCCTGCGTTATCCCCTGATTCTGTGGATAACCGT (SEQ ID NO. 25) |
| chrXI | CCACAAGTAAAGCTCGTTGACCAGTTGATCAGTTGAGGGGGGTACACACGACTAGCGCTTTCAGATATTAAA |
| 3'hom | AAGTTTAGATGTAGGTTTTAGCGGTAACAGTTATATAAATCGTGTTTCTTCTCTTGATGAAACAAAAAAATGCT |
| | AGAAAAACTTTGTCGTTTCTTACTTTTGGTGCGCTTTGCAGTTTTCGTGGCTAGACTTAGAATCATTTCTCCTCA |
| | GATTTCTTGATTAAAGTTTGGTGCGAAGCCCTACTCTAACATTGGTGTTCTTCTTTTCATTCACGCAAGTTAAGT |
| | CCAGGAAGGTGAGCAAATGCTCATCCTTCTGTTCATGCGTGACGGCTGAATTATCCTTATCTGGCGTACCCGT |
| | GCAGCCGTTTCCGTGCCTCGGTTCCTCCGAGATATCCTTAGGGACCGCCAGGGACCATGATTGCGTCAACTGT |
| | TGTCACCGCTCCAGAGGATCCTCTGTAACCTTTTCAACCAT (SEQ ID NO. 26) |
| HygR | AGCTTGCCTCGTCCCCGCCGGGTCACCCGGCCAGCGACATGGAGGCCCAGAATACCCTCCTTGACAGTCTTGA |
| | CGTGCGCAGCTCAGGGGCATGATGTGACTGTCGCCCGTACATTTAGCCCATACATCCCCATGTATAATCATTT |
| | GCATCCATACATTTTGATGGCCGCACGGCGCGAAGCAAAAATTACGGCTCCTCGCTCCAGACCTGCGAGCAG |
| | GGAAACGCTCCCCTCACAGACGCGTTGAATTGTCCCCACGCCGCGCCCCTGTAGAGAAATATAAAAGGTTAG |
| | GATTTGCCACTGAGGTTCTTCTTTCATATACTTCCTTTTAAAATCTTGCTAGGATACAGTTCTCACATCACATCC |
| | GAACATAAACAAAAATGGGTAAAAAGCCTGAACTCACCGCGACGTCTGTCGAGAAGTTTCTGATCGAAAAGT |
| | TCGACAGCGTGTCCGACCTGATGCAGCTCTCGGAGGGCGAAGAATCTCGTGCTTTCAGCTTCGATGTAGGAG |
| | GGCGTGGATATGTCCTGCGGGTAAATAGCTGCGCCGATGGTTTCTACAAAGATCGTTATGTTTATCGGCACTT |
| | TGCATCGGCCGCGCTCCCGATTCCGGAAGTGCTTGACATTGGGGAATTTAGCGAGAGCCTGACCTATTGCAT |
| | CTCCCGCCGTGCACAGGGTGTCACGTTGCAAGACCTGCCTGAAACCGAACTGCCCGCTGTTCTGCAACCGGTC |
| | GCGGAGGCCATGGATGCGATCGCTGCGGCCGATCTTAGCCAGACGAGCGGGTTCGGCCCATTCGGACCGCA |
| | AGGAATCGGTCAATACACTACATGGCGTGATTTCATATGCGCGATTGCTGATCCCCATGTGTATCACTGGCAA |
| | ACTGTGATGGACGACACCGTCAGTGCGTCCGTCGCGCAGGCTCTCGATGAGCTGATGCTTTGGGCCGAGGAC |
| | TGCCCCGAAGTCCGGCACCTCGTGCACGCGGATTTCGGCTCCAACAATGTCCTGACGGACAATGGCCGCATA |
| | ACAGCGGTCATTGACTGGAGCGAGGCGATGTTCGGGGATTCCCAATACGAGGTCGCCAACATCTTCTTCTGG |
| | AGGCCGTGGTTGGCTTGTATGGAGCAGCAGACGCGCTACTTCGAGCGGAGGCATCCGGAGCTTGCAGGATC |
| | GCCGCGGCTCCGGGCGTATATGCTCCGCATTGGTCTTGACCAACTCTATCAGAGCTTGGTTGACGGCAATTTC |
| | GATGATGCAGCTTGGGCGCAGGGTCGATGCGACGCAATCGTCCGATCCGGAGCCGGGACTGTCGGGCGTAC |
| | ACAAATCGCCCGCAGAAGCGCGGCCGTCTGGACCGATGGCTGTGTAGAAGTACTCGCCGATAGTGGAAACC |
| | GACGCCCCAGCACTCGTCCGAGGGCAAAGGAATAAAGTAACTGACAATAAAAAGATTCTTGTTTTCAAGAAC |
| | TTGTCATTTGTATAGTTTTTTTATATTGTAGTTGTTCTATTTTAATCAAATGTTAGCGTGATTTATATTTTTTTTC |
| | GCCTCGACATCATCTGCCCAGATGCGAAGTTAAGTGCGCAGAAAGTAATATCATGCGTCAATCGTATGTGAAT |
| | GCTGGTCGCTATACTG (SEQ ID NO. 27) |
| TRP1 | ACCAATCAGTAAAAATCAACGGTTAACGACATTACTATATATATAATATAGGAAGCATTTAATAGAACAGCAT |
| 5'hom | CGTAATATATGTGTACTTTGCAGTTATGACGCCAGATGGCAGTAGTGGAAGATATTCTTTATTGAAAAATAGC |
| | TTGTCACCTTACGTACAATCTTGATCCGGAGCTTTTCTTTTTTTGCCGATTAAG (SEQ ID NO. 28) |
| TRP1 | CAGGAAAATATACATCGCAGGGGGTTGACTTTTACCATTTCACCGCAATGGAATCAAACTTGTTGAAGAGAAT |
| 3'hom | GTTCACAGGCGCATACGCTACAATGACCCGATTCTTGCTAGCCTTTTCTCGGTCTTGCAAACAACCGCCGGCA |
| | GCTTAGTATATAAATACACATGTACATACCTCTCTCCGTATCCTCGTAATCATTTTCTTGTATTTATCGTCTTTTC |
| | GCTGTAAAAACTTTATCACACTTATCTCAAATACACTTATTAACCGC (SEQ ID NO. 29) |
| TRP1 | AATTCGGTCGAAAAAAGAAAAGGAGAGGGCCAAGAGGGAGGGCATTGGTGACTATTGAGCACGTGAGTAT |
| | ACGTGATTAAGCACACAAAGGCAGCTTGGAGTATGTCTGTTATTAATTTCACAGGTAGTTCTGGTCCATTGGT |
| | GAAAGTTTGCGGCTTGCAGAGCACAGAGGCCGCAGAATGTGCTCTAGATTCCGATGCTGACTTGCTGGGTAT |
| | TATATGTGTGCCCAATAGAAAGAGAACAATTGACCCGGTTATTGCAAGGAAAATTTCAAGTCTTGTAAAAGC |
| | ATATAAAAATAGTTCAGGCACTCCGAAATACTTGGTTGGCGTGTTTCGTAATCAACCTAAGGAGGATGTTTTG |
| | GCTCTGGTCAATGATTACGGCATTGATATCGTCCAACTGCATGGAGATGAGTCGTGGCAAGAATACCAAGAG |
| | TTCCTCGGTTTGCCAGTTATTAAAAGACTCGTATTTCCAAAAGACTGCAACATACTACTCAGTGCAGCTTCACA |
| | GAAACCTCATTCGTTTATTCCCTTGTTTGATTCAGAAGCAGGTGGGACAGGTGAACTTTTGGATTGGAACTCG |
| | ATTTCTGACTGGGTTGGAAGGCAAGAGAGCCCCGAAAGCTTACATTTTATGTTAGCTGGTGGACTGACGCCA |
| | GAAAATGTTGGTGATGCGCTTAGATTAAATGGCGTTATTGGTGTTGATGTAAGCGGAGGTGTGGAGACAAAT |
| | GGTGTAAAAGACTCTAACAAAATAGCAAATTTCGTCAAAAATGCTAAGAAATAGGTTATTACTGAGTAGTATT |
| | TATTTAAGTATTGTTTGTGCACTTGCCTGCAGGCCTTTTGAAAAGCAAGCATAAAAGATC (SEQ ID NO. 30) |

### Example 1: Improving the Yield of TAL Via NLSs SV40 and SV4040

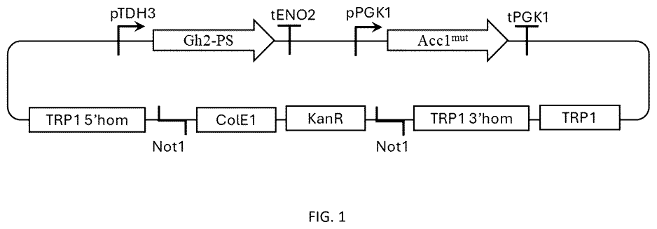

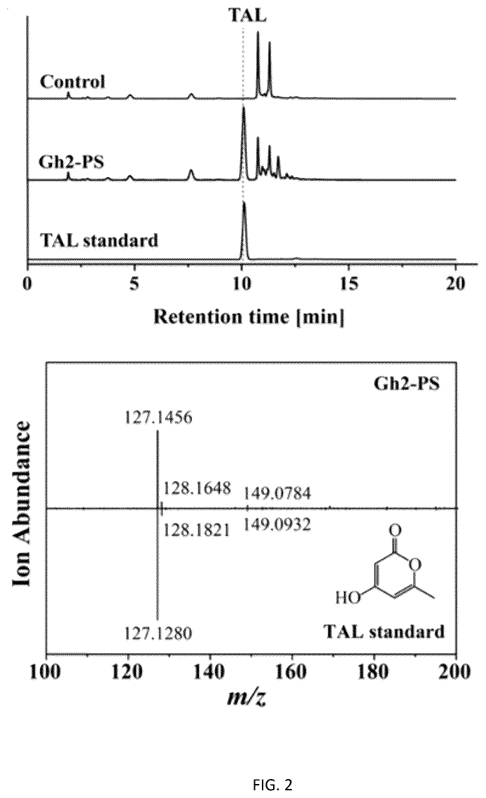

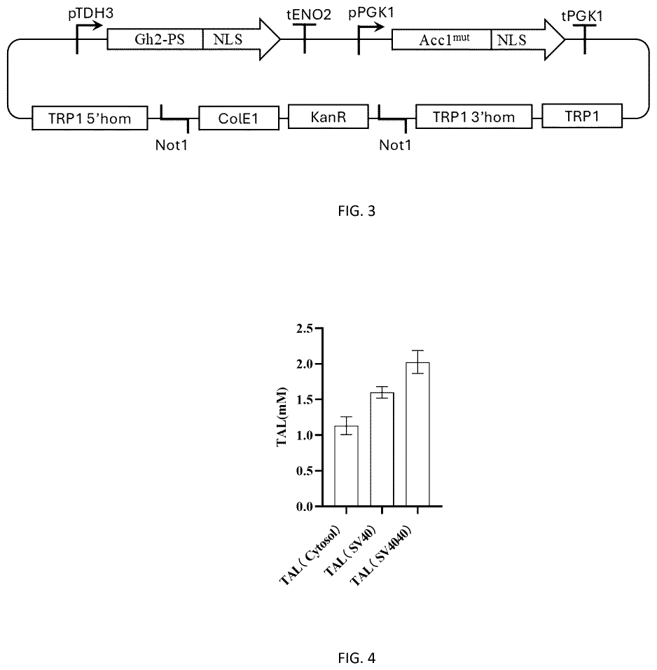

(1) Construction and verification of TAL biological metabolic pathway: Key enzymes required for TAL production include Acc1^{mut} (with a nucleotide sequence as set forth in SEQ ID NO. 3) and Gh2-PS (with a nucleotide sequence as set forth in SEQ ID NO. 4). First, homologous recombination was performed in Escherichia coli JM109 using ABclonal 2× MultiF Seamless Assembly Mix to construct a yeast expression cassette, as shown in FIG. 1. The plasmid was linearized with a restriction endonuclease NotI and then transformed into yeast CENPK2-1D. The constructed recombinant S. cerevisiae was cultured on a selective SC agar plate at 30° C. for 48 h for screening a correct engineered strain. As shown in FIG. 2, the results of verification via liquid chromatography-mass spectrometry confirmed that the engineered strain could heterologously produce TAL, indicating the successful construction of a cytoplasmic metabolic pathway. This engineered strain was named TAL (Cytosol) as a cytoplasmic pathway engineered strain.
(2) NLSs SV4040 (with a nucleotide sequence as set forth in SEQ ID NO. 1) and SV40 (with a nucleotide sequence as set forth in SEQ ID NO. 2) were respectively fused to C-terminals of the key enzymes Acc1^{mut} and Gh2-PS: Based on the expression cassette constructed in step (1), NLSs were added to C-terminals of Acc1^{mut} and Gh2-PS via overlapping extension of PCR, and the resulting constructs were transformed into E. coli JM109 to obtain a novel yeast expression cassette (as shown in FIG. 3, with involved sequences listed in Table 1). The plasmid was linearized with the restriction endonuclease NotI and then transformed into yeast CENPK2-1D, such that the two key enzymes in the yeast CENPK2-1D were localized to the nucleus as nuclear pathway engineered strains, and were named TAL (SV40) and TAL (SV4040), respectively.
(3) Fermentation for performance verification: The three genetically engineered yeast strains obtained in step (1) and step (2) were inoculated into 2 mL of selective SC medium in a 24-well deep-well plate and cultured at 30° C. and 220 rpm for 24 h. Subsequently, 100 μl of a seed culture was inoculated into a new 24-well deep-well plate containing 2 mL of YTD medium per well and enabled to grow at 30° C. and 220 rpm for 48 h. First, the cell density (OD₆₀₀) was measured using an ultraviolet and visible spectrophotometer 759S. The culture was then centrifuged at 12,000 rpm for 5 min. Finally, the supernatant was filtered through a 0.22 μm filter membrane and transferred to HPLC vials for liquid phase analysis. As shown in FIG. 4, the yield of TAL obtained via the nuclear pathway engineered strain TAL (SV40) was 41.2% higher than that of TAL obtained via the cytoplasmic pathway engineered strain TAL (Cytosol), indicating that the NLS successfully improved TAL synthesis efficiency in yeast. Furthermore, the yield of TAL obtained via the nuclear pathway engineered strain TAL (SV4040) was 26.7% higher than that of TAL obtained via the nuclear pathway engineered strain TAL (SV40), indicating that the novel NLS mutant SV4040 exhibited more advantages in improving the synthesis efficiency.

### Example 2: Improving the Yield of Flaviolin Via NLSs SV40, SV4040 and cMyc

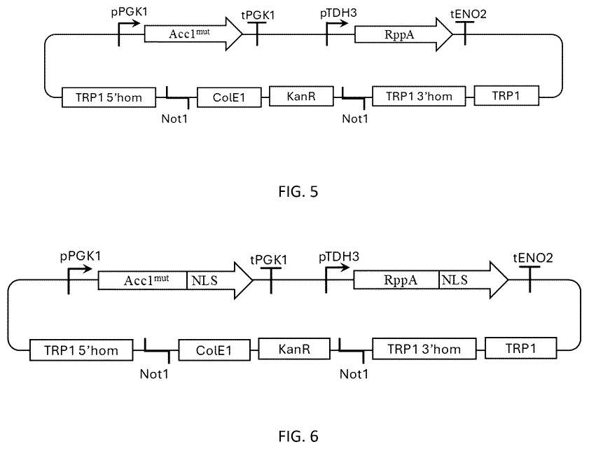

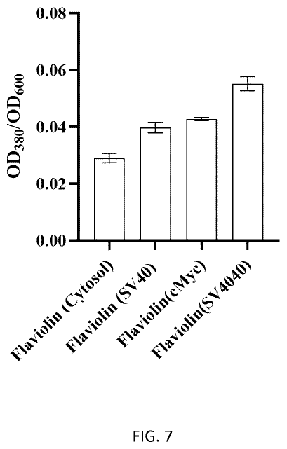

(1) Construction and verification of flaviolin biological metabolic pathway: Key enzymes required for flaviolin production include Acc1^{mut} (with a nucleotide sequence as set forth in SEQ ID NO. 3) and RppA (with a nucleotide sequence as set forth in SEQ ID NO. 10). First, homologous recombination was performed in E. coli JM109 using ABclonal 2× MultiF Seamless Assembly Mix to construct a yeast expression cassette (as shown in FIG. 5, with involved sequences listed in Table 1). The plasmid was linearized with a restriction endonuclease NotI and then transformed into yeast CENPK2-1D. The constructed recombinant S. cerevisiae was cultured on a selective SC agar plate at 30° C. for 48 h for screening a correct engineered strain. This engineered strain was named Flaviolin (Cytosol) as a cytoplasmic pathway engineered strain.
(2) NLSs SV4040 (with a nucleotide sequence as set forth in SEQ ID NO. 1), SV40 (with a nucleotide sequence as set forth in SEQ ID NO. 2), and cMyc (with a nucleotide sequence as set forth in SEQ ID NO. 11) were respectively fused to C-terminals of the key enzymes Acc1^{mut} and RppA: Based on the expression cassette constructed in step (1), NLSs were added to C-terminals of Acc1^{mut} and RppA via overlapping extension of PCR, and the resulting constructs were transformed into E. coli JM109 to obtain a novel yeast expression cassette (as shown in FIG. 6). The plasmid was linearized with the restriction endonuclease NotI and then transformed into yeast CENPK2-1D, such that the two key enzymes in the yeast CENPK2-1D were localized to the nucleus as nuclear pathway engineered strains, and were named Flaviolin (SV40), Flaviolin (SV4040) and Flaviolin (cMyc), respectively.
(3) Fermentation for performance verification: The four genetically engineered yeast strains obtained in step (1) and step (2) were inoculated into 2 mL of selective SC medium in a 24-well deep-well plate and cultured at 30° C. and 220 rpm for 24 h. Subsequently, 100 μL of a seed culture was inoculated into a new 24-well deep-well plate containing 2 mL of YTD medium per well and enabled to grow at 30° C. and 220 rpm for 48 h. First, the cell density (OD 600) was measured using an ultraviolet and visible spectrophotometer 759S. The culture was then centrifuged at 12,000 rpm for 5 min. The supernatant was taken and an absorbance value thereof at OD₃₈₀ was detected by a microplate reader as the yield of flaviolin. As shown in FIG. 7, the yields of flaviolin obtained via the nuclear pathway engineered strains Flaviolin (SV40) and Flaviolin (cMyc) were respectively 36.8% and 47.4% higher than that of flaviolin obtained via the cytoplasmic pathway engineered strain Flaviolin (Cytosol), indicating that the NLSs successfully improved flaviolin synthesis efficiency in yeast. Furthermore, the yield of flaviolin obtained via the nuclear pathway engineered strain Flaviolin (SV4040) was 39% and 29% higher than those of flaviolin obtained via the nuclear pathway engineered strains Flaviolin (SV40) and Flaviolin (cMyc), indicating that the novel NLS mutant SV4040 exhibited more advantages in improving the synthesis efficiency.

### Example 3: Improving the Yield of HT Via NLS SV4040

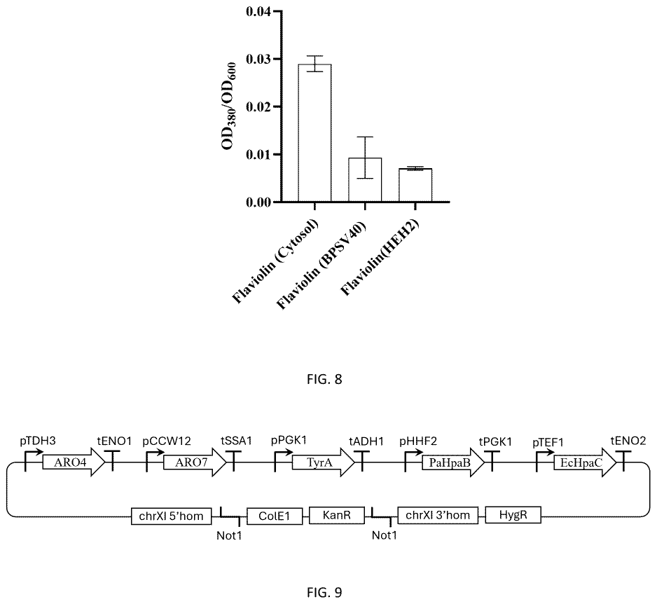

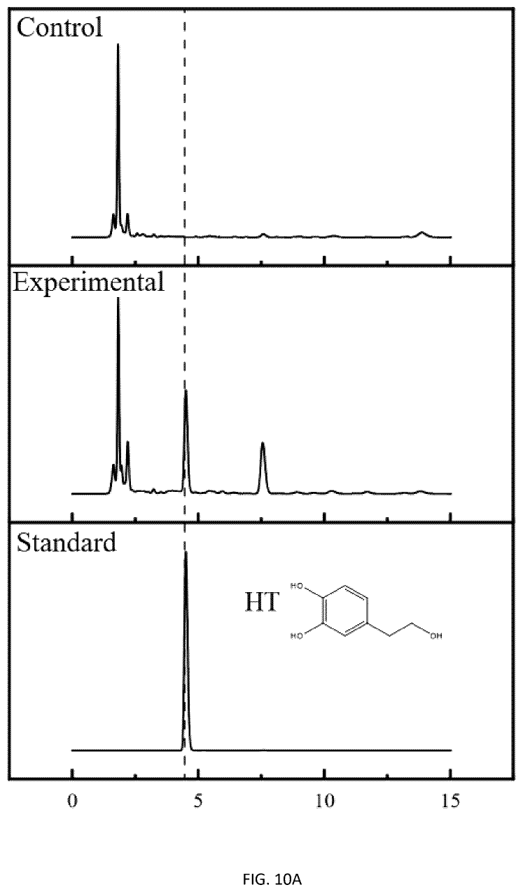

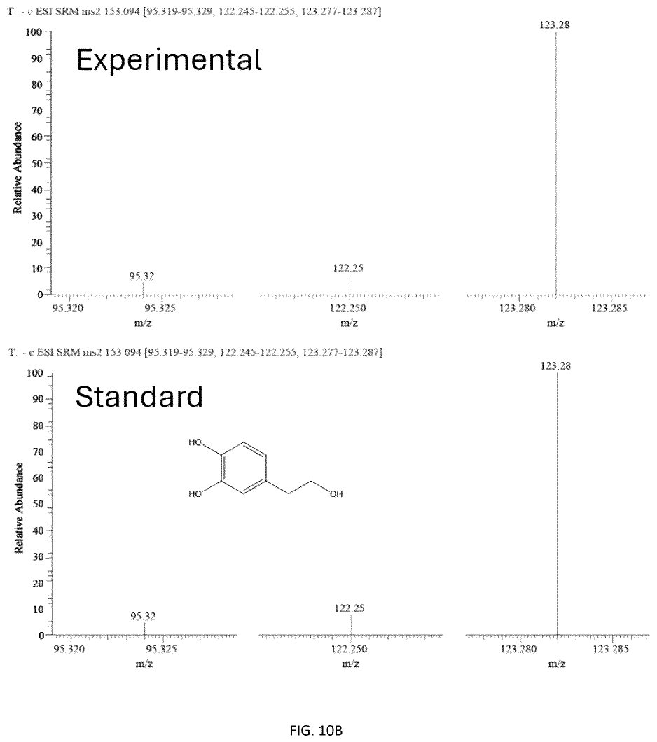

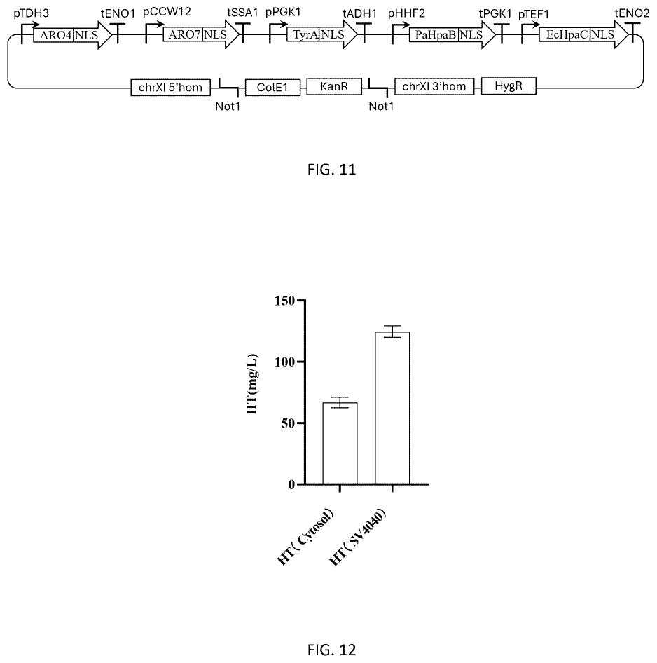

(1) Construction and verification of HT biological metabolic pathway: Key enzymes required for HT production include ARO4 (with a nucleotide sequence as set forth in SEQ ID NO. 5), ARO7 (with a nucleotide sequence as set forth in SEQ ID NO. 6), TyrA (with a nucleotide sequence as set forth in SEQ ID NO. 7), PaHpaB (with a nucleotide sequence as set forth in SEQ ID NO. 8) and EcHpaC (with a nucleotide sequence as set forth in SEQ ID NO. 9). First, homologous recombination was performed in E. coli JM109 using ABclonal 2× MultiF Seamless Assembly Mix to construct a yeast expression cassette (as shown in FIG. 9, with involved sequences listed in Table 1). The plasmid was linearized with NotI and then transformed into yeast CENPK2-1D. The constructed recombinant S. cerevisiae was cultured on a YTD agar plate medium containing 200 μg/ml hygromycin B at 30° C. for 48 h for screening a correct engineered strain. As shown in FIG. 10, the results of verification via liquid chromatography-mass spectrometry confirmed that the engineered strain could heterologously produce HT, indicating the successful construction of a cytoplasmic metabolic pathway. This engineered strain was named HT (Cytosol) as a cytoplasmic pathway engineered strain.
(2) SV4040 (with a nucleotide sequence as set forth in SEQ ID NO. 1) was separately fused to C-terminals of the key enzymes ARO4, ARO7, TyrA, PaHpaB, and EcHpaC: Based on the expression cassette constructed in step (1), NLSs were added to C-terminals of ARO4, ARO7, TyrA, PaHpaB, and EcHpaC via overlapping extension of PCR, and the resulting constructs were transformed into E. coli JM109 to obtain a novel yeast expression cassette, with results as shown in FIG. 11. The plasmid was linearized with the NotI and then transformed into yeast CENPK2-1D, such that the five key enzymes in the yeast CENPK2-1D were localized to the nucleus as nuclear pathway engineered strains, and were named HT (SV4040).
(3) Fermentation for performance verification: The two genetically engineered yeast strains obtained in step (1) and step (2) were inoculated into 2 mL of YTD medium containing 200 μg/ml hygromycin B in a 24-well deep-well plate and cultured at 30° C. and 220 rpm for 24 h. Subsequently, 100 μl of a seed culture was inoculated into a new 24-well deep-well plate containing 2 mL of YTD medium per well and enabled to grow at 30° C. and 220 rpm for 48 h. First, the cell density (OD₆₀₀) was measured using an ultraviolet and visible spectrophotometer 759S. The culture was then centrifuged at 12,000 rpm for 5 min. Finally, the supernatant was filtered through a 0.22 μm filter membrane and transferred to HPLC vials for liquid phase analysis. As shown in FIG. 12, the yield of HT obtained via the nuclear pathway engineered strain HT (SV4040) was 103% higher than that of HT obtained via the cytoplasmic pathway engineered strain HT (Cytosol), indicating that the NLS successfully improved HT synthesis efficiency in yeast.

### Comparative Example 1

The specific embodiment was the same as that of Example 2, except that the NLS SV40 was separately replaced with BPSV40 (with a nucleotide sequence as set forth in SEQ ID NO. 12) and HEH2 (with a nucleotide sequence as set forth in SEQ ID NO. 13). Recombinant strains Flaviolin (BPSV40) and Flaviolin (HEH2) were separately constructed and fermented according to the method of Example 2. The results are shown in FIG. 8. The yields of the two nuclear pathway engineered strains are significantly lower than that of the cytoplasmic pathway engineered strain, indicating that only the NLS with a binding strength to a transport protein Kap60 similar to a binding strength of SV40 to the transport protein Kap60 can improve the biosynthetic efficiency, and the new mutant SV4040 has the best effect.

Although the exemplary examples of the present disclosure have been provided above, they are not intended to limit the present disclosure. Those skilled in the art will appreciate that various changes and modifications might be made without departing from the spirit and scope of the present disclosure. Therefore, the protection scope of the present disclosure shall be defined by the claims.

What is claimed is:

1. A nuclear localization sequence (NLS), wherein the nucleotide sequence of the NLS is set forth in SEQ ID NO: 1.

2. A biological material comprising the NLS according to claim 1.

3. The biological material according to claim 2, wherein the biological material is a nucleic acid construct.

4. The biological material according to claim 3, wherein the nucleic acid construct comprises at least one NLS and at least one gene related to a biological metabolic pathway; and the NLS is linked upstream or downstream of the gene related to the biological metabolic pathway for guiding a directional localization of a gene expression product to a host cell nucleus.

5. The biological material according to claim 4, wherein a protein encoded by the gene related to the biological metabolic pathway is involved in a synthetic pathway of a target metabolite in a microorganism.

6. The biological material according to claim 4, wherein the host is a eukaryotic microorganism; and the eukaryotic microorganism comprises Saccharomyces cerevisiae.

7. The biological material according to claim 2, wherein the biological material is recombinant S. cerevisiae, comprising a gene localized under the guidance of the NLS.

8. The biological material according to claim 7, comprising any one of (a) to (c):
(a) the recombinant S. cerevisiae comprises an Acc1 gene with a nucleotide sequence as set forth in SEQ ID NO: 3 and a 2-Pyrone synthase (Gh2-PS) gene with a nucleotide sequence set forth in SEQ ID NO: 4, and 3′ ends of the Acc1 gene and the Gh2-PS gene are separately linked to the NLS;
(b) the recombinant S. cerevisiae comprises the Acc1 gene with the nucleotide sequence set forth in SEQ ID NO: 3 and an RppA gene with the nucleotide sequence as set forth in SEQ ID NO: 10, and 3′ ends of the Acc1 gene and the RppA gene are separately linked to the NLS; and
(c) the recombinant S. cerevisiae comprises an ARO4 gene with the nucleotide sequence set forth in SEQ ID NO: 5, an ARO7 gene with the nucleotide sequence set forth in SEQ ID NO: 6, a TyrA gene with the nucleotide sequence as set forth in SEQ ID NO: 7, a PaHpaB gene with the nucleotide sequence as set forth in SEQ ID NO: 8 and an EcHpaC gene with the nucleotide sequence as set forth in SEQ ID NO: 9, and 3′ ends of the ARO4 gene, the ARO7 gene, the TyrA gene, the PaHpaB gene and the EcHpaC gene are separately linked to the NLS.

9. A method for improving biosynthetic efficiency, comprising following steps:
(a) constructing a recombinant nucleic acid construct, wherein the construct comprises: at least one nuclear localization sequence (NLS), and at least one gene encoding a key enzyme in a synthetic pathway of a target metabolite; the NLS is operably linked upstream or downstream of the gene; and the nucleotide sequence of the NLS is set forth in SEQ ID NO: 1;
(b) introducing the recombinant nucleic acid construct in (a) into a eukaryotic microbial host cell;
(c) culturing the host cell in (b) under suitable conditions to enable the expression and localization of the key enzyme within a cell nucleus; and
(d) collecting the target metabolite.

10. The method according to claim 9, wherein the target metabolite comprises products of the metabolic pathways of triacetic acid lactone (TAL), mevalonic acid (MVA), tryptophol, methyl anthranilate (Me-AA), 2-phenylethanol (2-PE), tyrosol, or hydroxytyrosol (HT).
